(19) United States
(12) Patent Application Publication
(10) Pub. No.: US 2022/0351823 A1
(43) Pub. Date: Nov. 3, 2022

(54) STEPS EXPRESSED RELATIVE TO BODY FAT MASS PREDICTS BODY COMPOSITION AND CARDIOMETABOLIC RISK IN ADULTS EATING AD LIBITUM

(71) Applicant: Kennesaw State University Research And Service Foundation, Inc., Kennesaw, GA (US)

(72) Inventor: Robert Buresh, Dallas, GA (US)

(21) Appl. No.: 17/733,275

(22) Filed: Apr. 29, 2022

Related U.S. Application Data
(60) Provisional application No. 63/181,483, filed on Apr. 29, 2021.

(51) Int. Cl. G16H 20/30; G16H 10/60

(52) U.S. Cl. CPC G16H 20/30; G16H 10/60

(57) ABSTRACT

A method for customized activity level recommendations, the method comprising: receiving via an interface, at least one body health indicator associated with a user; determining, based at least on the body health indicator, a fat mass metric of the user and target weight and body composition of the user; determining, based on at least the fat mass metric and using a recommendation algorithm comprising a set of rules, a custom physical activity threshold for the user; and outputting an indication of the custom physical activity threshold.

### CROSS-REFERENCE TO RELATED APPLICATIONS

This patent application claims priority to and the benefit of U.S. Provisional Application No. 63/181,483 filed Apr. 29, 2021, which is hereby incorporated herein in its entirety.

### BACKGROUND

Many adults struggle with body weight throughout their lives, and, though many have been successful in managing their weight through exercise, a larger number of people have been unable to achieve and sustain weight loss via exercise.

Generally, physical activity/exercise for weight management is prescribed in a non-individualized way, and recommendations are very broad.

Improvements are needed.

### SUMMARY

The present disclosure relates to systems and methods for customized activity level recommendations to maintain or improve human health. Systems and methods may comprise receiving via an interface, at least one body health indicator associated with a user; determining, based at least on the body health indicator, a fat mass metric of the user; determining, based on at least the fat mass metric and using a recommendation algorithm comprising a set of rules, a custom physical activity threshold for the user; and outputting an indication of the custom physical activity threshold.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method for customized activity level recommendations. The method also includes receiving via an interface, at least one body health indicator associated with a user; determining, based at least on the body health indicator, a fat mass metric of the user and a weight/body fatness target of the user; determining, based on at least the fat mass metric and using a recommendation algorithm may include a set of rules, a custom physical activity threshold for the user; and outputting an indication of the custom physical activity threshold. Other embodiments of this aspect include corresponding computer systems, apparatus, and computer programs recorded on one or more computer storage devices, each configured to perform the actions of the methods.

One general aspect includes a non-transitory computer readable storage medium having instructions stored thereon that. The non-transitory computer readable storage medium also includes receiving via an interface, at least one body health indicator associated with a user; determining, based at least on the body health indicator, a fat mass metric of the user and a weight/body fatness target of the user; determining, based on at least the fat mass metric and using a recommendation algorithm may include a set of rules, a custom physical activity threshold for the user; and outputting an indication of the custom physical activity threshold. Other embodiments of this aspect include corresponding computer systems, apparatus, and computer programs recorded on one or more computer storage devices, each configured to perform the actions of the methods.

One general aspect includes a device for executing a method for customized activity level recommendations an interface configured to: receive at least one body health indicator associated with a user; determine, based at least on the body health indicator, a fat mass metric of the user and a weight/body fatness target of the user; determine, based on at least the fat mass metric and using a recommendation algorithm may include a set of rules, a custom physical activity threshold for the user; and output an indication of the custom physical activity threshold. Other embodiments of this aspect include corresponding computer systems, apparatus, and computer programs recorded on one or more computer storage devices, each configured to perform the actions of the methods.

### BRIEF DESCRIPTION OF THE DRAWINGS

The following drawings show generally, by way of example, but not by way of limitation, various examples discussed in the present disclosure. In the drawings:

FIG. 1 shows the correlation between body fatness and the number of steps taken per kilogram of fat mass per day.

FIG. 2 shows the correlation between body fatness and the number of steps taken per day.

FIG. 3 shows the correlation between the ratio of total cholesterol to high density lipoprotein cholesterol (TC:HDL) and the number of steps taken per kilogram of fat mass per day.

FIG. 4 shows the correlation between the relative number of steps taken per day and the quantitative insulin sensitivity check index (QUICKI).

FIG. 5 depicts the settling point model of body weight regulation.

FIG. 6 depicts predicted vs. measured body composition.

### DETAILED DESCRIPTION

Many adults struggle with weight throughout their lives, and, though many have been successful in managing weight through exercise, a larger number of people have been unable to achieve and sustain weight loss via exercise. Step-count targets that can be expected to produce predictable changes in body weight may vastly increase the number of people who are able to successfully manage weight over their adult lives.

Common activity targets are not specific to the individual (e.g., accumulate 10,000 steps/day, expend 1,500 kcals in physical activity each week, or accumulate 420 min of moderate intensity physical activity each week), and, importantly, such targets do not take into account key influences of appetite and energy intake. The systems and methods described herein may address one or more of these shortcomings by leveraging a model (e.g., recommendation algorithm) that is customized based on body fat mass. The systems and methods described herein may address one or more of these shortcomings by determining physical activity levels, such as step count targets, that are individualized, and that account for current body fat mass and target body fat mass, as well as fat-free mass, which is a key determinant of appetite and energy intake.

A method for customized activity level recommendations is described herein. The method may include receiving via an interface, at least one body health indicator associated with a user. The method may further include determining, based at least on the body health indicator, a fat mass metric of the user and a weight/body fatness target of the user. The method may then determine, based on at least the fat mass metric and using a recommendation algorithm comprising a set of rules, a custom physical activity threshold for the user; and output an indication of the custom physical activity threshold.

The at least one body health indicator may be a user's sex, body mass, body fat mass, fat-free mass, body fatness percentage, visceral fat mass, total cholesterol level, high-density lipoprotein cholesterol level, low-density lipoprotein cholesterol level, insulin level, glucose level, triglyceride level, glycosylated hemoglobin level, a cardiometabolic risk factor, an energy intake, an energy expenditure or combinations thereof. The at least one body health may be a user's target body mass, target body fat mass, target fat-free mass, target body fatness percentage, target visceral fat mass, target total cholesterol level, target high-density lipoprotein cholesterol level, target low-density lipoprotein cholesterol level, target insulin level, target glucose level, target triglyceride level, target glycosylated hemoglobin level, target cardiometabolic risk factor, target energy intake, or target energy expenditure or some combination thereof.

The custom physical activity threshold comprises a number of steps taken per day. The custom physical activity threshold comprises a number of steps taken per day per unit of body fat mass, and based thereon, a total daily step count.

The interface may be an activity tracking device. The activity tracking device may incorporate the systems and methods described herein to assist users with selecting a physical activity level that may yield a targeted change in weight/body composition.

The method may include using regression modeling to predict body fatness from daily step counts. The prevalence of overweight and obesity among adults in the U.S. continues to rise, and the associated public health consequences are costly and severe. Excess body fatness is independently associated with increased rates of diabetes, hypertension, dyslipidemia, coronary heart disease, and other chronic conditions. In addition, it is estimated that approximately 34% of U.S. adults have metabolic syndrome, a condition characterized by a combination of central obesity, dyslipidemia, glucose intolerance, and hypertension. Furthermore, recent research shows that, although overall cancer diagnoses and mortality rates in the US are declining, obesity-related cancers are on the rise. A persistent increase in the prevalence of obesity among adults has continued uninterrupted for 40 years, increasing from approximately 14% in 1980 to over 40% today, in spite of myriad efforts to arrest it. Projection of recent trends has led to the troubling prediction that prevalence of adults diagnosed with type 2 diabetes will more than double by 2060.

While the causes of increasing obesity and its comorbidities (e.g., cardiometabolic risk [CMR] factors) are multifactorial, in basic terms, increasing numbers of adults are now exhibiting a chronic energy surplus — that is, a state in which energy intake via diet is consistently greater than the total energy expended (the sum of energy expended in resting metabolism, physical activity, and diet-induced thermogenesis). Numerous research efforts have been undertaken to identify factors that have changed since 1980 that contribute to this epidemic, with widely varying conclusions. Several studies have reported concurrent reductions in various forms of physical activity over the same time, suggesting that diminishing amounts of physical activity are contributing to the increasing prevalence of overweight and obesity. However, although the importance of physical activity (PA) in weight management is apparent in that most weight management guidelines include recommendations for PA, the influence of PA/exercise in weight control interventions is often trivial, and several recently published reviews have concluded that PA contributes little to weight management interventions relative to dietary restriction. While energy balance, and factors that influence it, are complex, it is apparent that many PA/exercise interventions for weight management are insufficient to induce a sustained energy deficit.

Overweight and obesity have been mechanistically linked to type 2 diabetes, cardiovascular disease, and several forms of cancer. Several CMR factors are associated with excess body fatness (e.g., insulin resistance, plasma lipids), as reflected in the diagnostic criteria for metabolic syndrome (abdominal obesity, hypertension, dyslipidemia, and elevated plasma glucose). Understanding variables that are influential of body fatness and CMR profiles is, therefore, of increasing importance. The present disclosure relates to using current and target body weight and composition to develop a step count target that will result in predictable changes in body weight and body fat percentage over several months while eating ad libitum.

### EXAMPLES

A study was carried out at a university. Participants were recruited from among a university community (faculty, staff, students, and nearby residents) in the southeast U.S. via email and posted announcements. The primary inclusion criterion was age (between 19-40 years old), while exclusion criteria included the presence of any diagnosed chronic disease, current involvement in commercial weight management program, and being on prescription medications other than birth control. After providing informed consent, participants made an appointment to arrive to the exercise physiology laboratory between 6:00 a.m. and 9:00 a.m. in a fasted condition (no food or beverage except water for 10-12 hours prior to arrival). They were asked to avoid vigorous exercise on the day prior to laboratory appointments. A total of 34 adults made a laboratory visit in which height, weight, body composition, and cardiometabolic risk factors were quantified. Participants wore accelerometers for 21-28 days, then returned to the laboratory for a second body composition assessment. Changes in weight and body composition were used to quantify energy balance, and data derived from accelerometers provided markers of physical activity and sedentariness.

Of the markers of physical activity measured, daily step counts expressed relative to fat mass was most strongly and consistently associated with body fatness and cardiometabolic risk status. Step counts expressed relative to fat mass were strongly associated with body composition and cardiometabolic risk in adults eating ad libitum.

The initial target was to recruit a total of 40 participants (20 men and 20 women), as a previous pilot study resulted in a strong inverse relationship between steps expressed relative to FM (STEPS·kg FM⁻¹·day⁻¹) and body fatness in men (n=12, r=−0.90) and women (n=34, r=−0.73). To achieve a correlational power of 0.90 (p=0.05), assuming a Pearson r correlation of 0.73, would require n=15; therefore, the systems and methods described herein targeted n=20 men, and n=20 women.

Urine samples were collected, and urine specific gravity was measured to provide an indication of hydration status. Height and weight were measured on a medical-grade scale and stadiometer (Tanita Corporation of America, Arlington Heights, Ill.). Bioelectric impedance analysis (BIA) was then conducted on an InBody 770 analyzer (Cerritos, Calif.). Thereafter, participants underwent a dual-energy x-ray absorptiometry (DEXA) scan to assess body fatness and fat distribution. The assessment was conducted in accordance with manufacturer's (iDXA, General Electric, Inc., Madison, Wis.) guidelines to quantify body fat percentage (BF), visceral fat area (VFA), fat mass (FM), and fat-free mass (FFM). VFA measurement was obtained by the iDXA software (enCORE version 13.60) using default android region boundaries.

Thereafter, participants provided a finger-stick blood sample. Fasting plasma lipids (total cholesterol [TC], high-density lipoprotein cholesterol [HDL], low-density lipoprotein cholesterol [LDL], and triglycerides [TG]) were measured using a Cholestech LDX analyzer (Alere, Orlando, Fla.). Plasma glucose (GLU) was measured in duplicate using a Medtronic Contour glucometer (Bayer, Pittsburgh, Pa.), with the average of the two readings recorded. Glycosylated hemoglobin (HbA1c) was also measured using finger-stick blood sample with a Siemens DCA Vantage (Malvern, Pa.) analyzer. In addition, blood was collected on Whatman 903 Protein Saver Cards (GE Healthcare Ltd., Cardiff, CF UK) and allowed to dry, and dry blood spots were analyzed later for plasma insulin (INS). Quantitative check index for insulin sensitivity (QUICKI) was calculated using fasting GLU and INS levels.

Participants were then provided instructions for the wearing of accelerometers. Each participant was provided with an Actigraph GT3x (Pensacola, Fla.) accelerometer and a wrist strap, and were instructed to wear the device for all of their waking hours, with the exception of time spent bathing or showering. All accelerometers were initialized using information describing the participant (e.g., age, height, weight, and sex of the participant). Accelerometers were worn on the non-dominant wrist, and participants were encouraged to live and eat normally, and to not alter their behaviors during the course of their involvement in the study. Follow-up appointments were made for 21-28 days later. During the follow-up laboratory visit, urine specific gravity was again measured, weight was measured on the same scale used in the first visit, and BIA and DEXA scans were again conducted. Accelerometers were collected, and data were downloaded using the ActiLife software (version 6.13.3). Activity was assessed in 10-sec epochs, and wear-time analysis was performed. Data derived from participants who wore accelerometers for fewer than an average of 12 hours per day (n=4) were eliminated from further analysis. Estimates of daily step counts, MVPA, and SED were recorded.

Body composition was determined using 4-compartment modeling. Body volume was determined using data derived from DEXA scans, and total body water was derived from BIA assessment. BF was determined as described previously. Energy balance for the study period was determined by quantifying the changes in FM and FFM across the study period, and the total differences were divided by the number of days between laboratory visits to determine the average daily energy balance. Participants were considered to be in a state of energy imbalance if the average daily energy balance showed a surplus or deficit of greater than 1,000 kcal·day⁻¹, and their data were not analyzed (n=3).

All statistical tests (descriptive statistics, Pearson r and Spearman's rho correlations, linear regression, and power regression) were performed using SPSS (IBM, Armonk, N.Y.), Version 26.

A total of 34 participants (19 females, 15 males) completed the study protocol, with average accelerometer wear times of at least 12 hours per day during their involvement while maintaining energy balance. Among those whose data were included, there was no correlation between accelerometer wear time and any expression of STEPS, and there were no differences in body composition, fat-free mass, FM, or weight between the two assessments. INS was not measured in one man as a result of a vaso-vagal response to the finger stick. In addition, HbA1c was not measured in two women due to reportedly low hemoglobin levels, and LDL and TG were not able to be measured in two men and two women due to range limitations of the measuring device (briefly, if TG are less than 45 mg·dl⁻¹, they cannot be accurately measured, and LDL then cannot be calculated with the Cholestech LDX). Urine specific gravity (USG) was normal (between 1.002 and 1.030) in all participants prior to each assessment. Descriptive data are provided in Table 1.

TABLE 1
Descriptive Statistics [mean ± standard deviation or median (interquartile range)].

| | All (n = 34) | | Women (n = 19) | | Men (n = 15) | |
|---|---|---|---|---|---|---|
| Age (yrs) | 31.4 ± 4.8 | | 30.6 ± 3.6 | | 32.0 ± 5.9 | |
| Height (cm) | 173.1 ± 9.8 | | 166.9 ± 8.2 | | 179.5 ± 5.3 | |
| Weight - Visit 2 (kg) | 83.2 ± 18.8 | | 79.2 ± 21.8 | | 88.2 ± 13.2 | |
| Body Fatness - Visit | 29.3 ± 13.1 | | 33.2 ± 12.9 | | 24.3 ± 11.8 | |
| 2 (%) | | | | | | |
| USG - Visit 1 | 1.018 ± 0.007 | | 1.018 ± 0.007 | | 1.017 ± 0.007 | |
| TC (mg · dl⁻¹) | 181.6 ± 31.6 | | 181.4 ± 33.4 | | 181.1 ± 30.1 | |
| HDL (mg · dl⁻¹) | 50.9 ± 15.5 | | 56.1 ± 16.2 | | 46.9 ± 13.9 | |
| LDL (mg · dl⁻¹) | 113.7 ± 26.8 | [n = 30] | 105.9 ± 26.7 | [n = 17] | 124.5 ± 18.2 | [n = 13] |
| TRIG (mg · dl⁻¹) | 79.0 | (46.5)* [n = 30] | 89.0 | (59.0)* [n = 17] | 79.0 (38.5)* | [n = 13] |

| | | | | | |
|---|---|---|---|---|---|
| A1c (%) | 5.4 ± 0.29 | [n = 32] | 5.3 ± 0.3 | [n = 17] | 5.4 ± 0.29 |

| GLU (mg · dl⁻¹) | 97.8 ± 9.1 | | 95.7 ± 7.3 | 100.8 ± 11.4 | |
|---|---|---|---|---|---|
| INS (mIU · L⁻¹) | 10.9 (4.04)* | [n = 33] | 11.0 (6.3)* | 10.9 ± 2.6 | [n = 14] |
| QUICKI | 0.33 ± 0.02 | [n = 33] | 0.33 ± 0.02 | 0.33 ± 0.01 | [n = 14] |

| | | | |
|---|---|---|---|
| Accelerometer Wear | 16.4 (6.03)* | 16.7 ± 3.3 | 17.1 ± 3.8 |
| Time (hr · day⁻¹) | | | |
| Energy Balance | 61.9 ± 490.8 | −90.5 ± 470.2 | 255.0 ± 458.9 |
| (kcal · day⁻¹) | | | |
| STEPS (steps · day⁻¹) | 10,753.1 ± 2198.1 | 10,128.9 ± 2229.0 | 11,543.7 ± 1950.0 |

*= significant Shapiro/Wilk test for non-normality (p < 0.05) and reported as Median (interquartile range); USG = urine specific gravity; TC = total cholesterol; HDL = high-density lipoprotein cholesterol; LDL = low-density lipoprotein cholesterol; TRIG—triglycerides; A1c = glycosylated hemoglobin; GLU = glucose; INS = insulin; HOMA-IR = homeostasis model assessment - insulin resistance; QUICKI—quantitative check index for insulin sensitivity

A key independent variable in this study was step count expressed relative to fat mass (STEPS·kg FM⁻¹·day⁻¹), and this variable was not normally distributed (significant Shapiro/Wilk test). Therefore, relationships with this indicator of physical activity were determined using the Spearman's rho.

Physical Activity and Body Fatness

Body fatness was strongly associated with several markers of physical activity in men and women, particularly when physical activity was expressed relative to FM (see Table 2).

TABLE 2
Correlations between Physical Activity and Body Fatness
[Pearson r or Spearman's Rho (p-level)].

| Variable | | Percent Body Fat | | Fat Mass | | Visceral Fat Mass | |
|---|---|---|---|---|---|---|---|
| SED | All | −0.16 | (0.378) | −0.10 | (0.583) | −0.11 | (0.527) |
| (min · day⁻¹) | Men | −0.08 | (0.769) | −0.07 | (0.809) | −0.17 | (0.540) |
| | Women | −0.24 | (0.317) | −0.13 | (0.606) | −0.05 | (0.851) |
| MVPA | All | −0.04 | (0.816) | −0.06 | (0.746) | 0.03 | (0.886) |
| (min · day⁻¹) | Men | 0.42 | (0.118) | 0.47 | (0.079) | 0.31 | (0.266) |
| | Women | −0.18 | (0.471) | −0.22 | (0.367) | −0.23 | (0.349) |
| STEPS · day⁻¹ | All | 0.01 | (0.975) | 0.02 | (0.918) | 0.08 | (0.646) |
| | Men | 0.25 | (0.361) | 0.27 | (0.331) | 0.17 | (0.542) |
| | Women | 0.06 | (0.824) | −0.01 | (0.966) | −0.13 | (0.607) |
| STEPS · kg⁻¹ · day⁻¹ | All | −0.47** | (0.005) | −0.57** | (<0.001) | −0.53** | (0.001) |
| | Men | −0.45 | (0.092) | −0.47 | (0.076) | −0.44 | (0.100) |
| | Women | −0.57* | (0.011) | −0.64** | (0.003) | −0.65** | (0.002) |
| STEPS · kg FFM⁻¹ · day⁻¹ | All | 0.23 | (0.198) | 0.06 | (0.774) | −0.08 | (0.642) |
| | Men | 0.41 | (0.134) | 0.37 | (0.174) | 0.33 | (0.226) |
| | Women | −0.03 | (0.904) | −0.13 | (0.610) | −0.26 | (0.291) |
| (Spearman's Rho) | All | −0.90** | (<0.001) | −0.92** | (<0.001) | −0.79** | (<0.001) |
| STEPS · kg FM⁻¹ · day⁻¹ | Men | −0.94** | (<0.001) | −0.92** | (<0.001) | −0.93** | (<0.001) |
| | Women | −0.88** | (<0.001) | −0.92** | (<0.001) | −0.88** | (<0.001) |

SED = sedentary time;
MVPA = moderate-to-vigorous physical activity;
FFM = fat-free mass;
*=p ≤ 0.05;
**= p ≤ 0.01

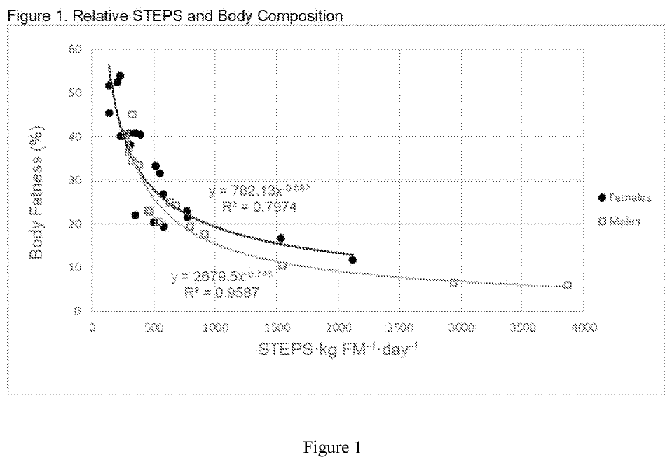

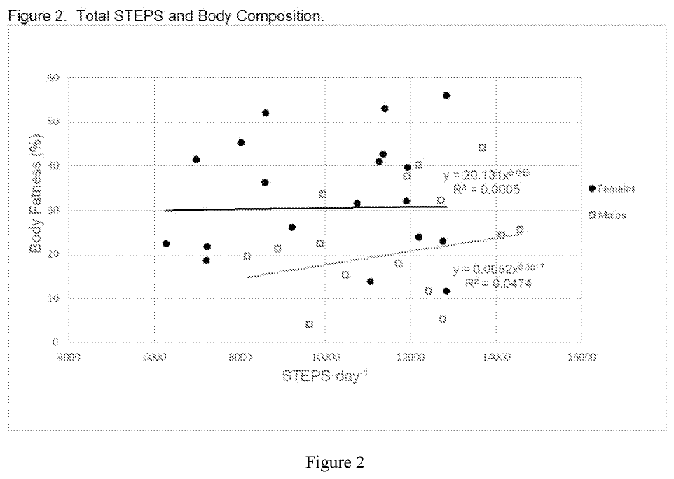

STEPS (STEPS·kg FM⁻¹·day ⁻¹) were inversely correlated with percent body fat, fat mass (FM), and visceral fat mass (VFM). STEPS was the strongest predictor of body fatness. Applying a power regression model (see FIG. 1), STEPS·kg FM⁻¹·day⁻¹ explained approximately 80% of the variability in body fat in women, and approximately 96% of the variability in body fat in men. There was no relationship between STEPS expressed in absolute terms (STEPS·day⁻¹) and body fatness (see FIG. 2).

FIG. 1 shows the body fat percentage as a function of daily steps taken relative to body fat mass. The x-axis units are steps per kilogram of fat mass (FM) per day. The figure shows this relationship for both male and female subjects. There is a strong correlation between the body fatness and the number of steps taken daily per kilogram of fat mass as demonstrated by the R² values close to one.

FIG. 2 shows the relationship between body fat composition and the number of daily steps taken. The figure shows that there is little to no correlation between these two values as indicated by the R² value less than 0.1.

Physical Activity and Cardiometabolic Risk Factors

Physical activity levels were associated with CMR profiles, particularly when physical activity was expressed relative to FM (see Table 3).

TABLE 3
Correlations between Physical Activity and Cardiometabolic Risk Factors [Pearson r or Spearman's Rho (p-level)].

| Variable | | TC:HDL | | HDL-C | | QUICKI | | GLU | |
|---|---|---|---|---|---|---|---|---|---|
| SED (min · day⁻¹) | All | −0.19 | (0.286) | −0.01 | (0.947) | 0.13 | (0.484) | −0.25 | (0.163) |
| | Men | −0.25 | (0.363) | −0.07 | (0.798) | 0.15 | (0.600) | −0.24 | (0.381) |
| | Women | −0.13 | (0.604) | 0.02 | (0.952) | 0.11 | (0.651) | −0.27 | (0.272) |
| MVPA (min · day⁻¹) | All | −0.01 | (0.960) | 0.01 | (0.941) | 0.36* | (0.037) | 0.20 | (0.256) |
| | Men | −0.06 | (0.820) | 0.16 | (0.570) | −0.16 | (0.587) | 0.43 | (0.108) |
| | Women | −0.06 | (0.800) | 0.02 | (0.921) | 0.58** | (0.009) | −0.03 | (0.913) |
| STEPS (steps · day⁻¹) | All | 0.06 | (0.731) | 0.01 | (0.956) | 0.22 | (0.212) | 0.28 | (0.111) |
| | Men | 0.03 | (0.922) | 0.02 | (0.931) | −0.17 | (0.560) | 0.26 | (0.357) |
| | Women | −0.04 | (0.857) | 0.17 | (0.484) | 0.440 | (0.060) | 0.18 | (0.464) |
| STEPS (steps · kg⁻¹ · day⁻¹) | All | −0.30 | (0.080) | 0.35* | (0.040) | 0.61** | (<0.001) | −0.25 | (0.152) |
| | Men | −0.36 | (0.187) | 0.41 | (0.125) | 0.42 | (0.140) | −0.37 | (0.178) |
| | Women | −0.32 | (0.189) | 0.35 | (0.146) | 0.69** | (0.001) | −0.23 | (0.345) |
| STEPS (steps · kg FFM⁻¹ · day⁻¹) | All | −0.05 | (0.765) | 0.18 | (0.315) | 0.25 | (0.156) | 0.09 | (0.604) |
| | Men | 0.21 | (0.462) | −0.05 | (0.862) | −0.35 | (0.222) | 0.38 | (0.124) |
| | Women | −0.07 | (0.766) | 0.17 | (0.495) | 0.52* | (0.024) | 0.09 | (0.715) |
| (Spearman's Rho) | All | −0.48** | (0.004) | 0.34* | (0.046) | 0.72** | (<0.001) | −0.57** | (<0.001) |
| STEPS (steps · kg FM⁻¹ · day⁻¹) | Men | −0.68** | (0.005) | 0.56* | (0.029) | 0.82** | (<0.001) | −0.87* | (<0.001) |
| | Women | −0.52* | (0.023) | 0.41 | (0.084) | 0.67** | (0.002) | −0.57* | (0.012) |

RMR = resting metabolic rate;
SED = sedentary time;
MVPA = moderate-to-vigorous physical activity;
FM = fat mass;
FFM = fat-free mass;
TC:HDL = total cholesterol:HDL cholesterol ratio;
HDL-C = high-density lipoprotein cholesterol;
QUICKI = quantitative insulin check index;
GLU = fasting glucose;
*= p ≤ 0.05;
**= p ≤ 0.01;

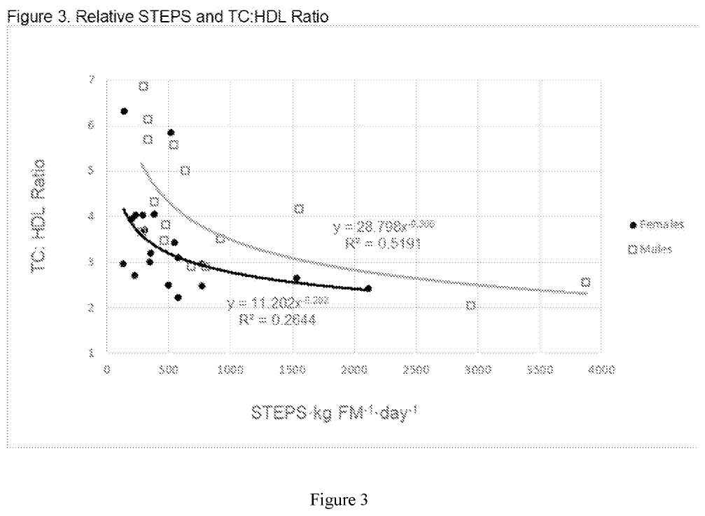

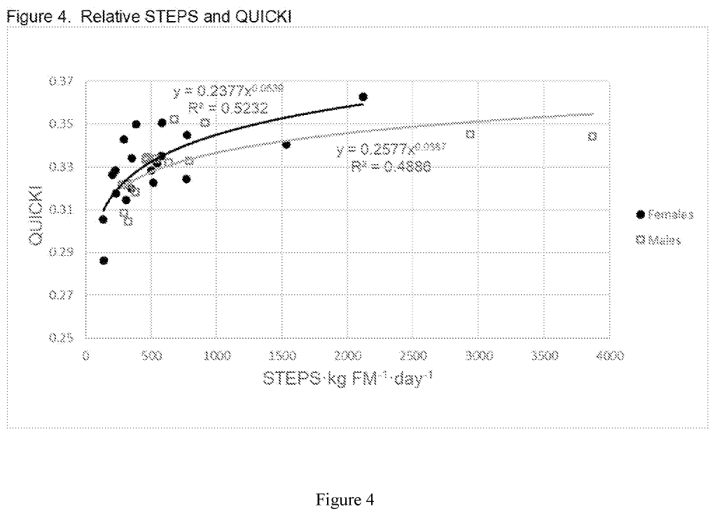

STEPS expressed relative to FM were most consistently correlated with TC:HDL ratio, HDL, QUICKI, and GLU in the full sample, as well as in men and women independently. Power regression analysis showed that STEPS·kg FM⁻¹·day⁻¹ was the best predictor of TC:HDL (R²=0.26 for women, R²=0.52 for men; see FIG. 3), as well as for QUICKI (R²=0.52 for women, R²=0.49 for men; see FIG. 4).

FIG. 3 shows STEPS relative to fat mass and total cholesterol:HDL ratio. FM=fat mass; TC=total cholesterol; HDL=high-density cholesterol.

FIG. 4 shows STEPS relative to fat mass and quantitative insulin sensitivity check index. FM=fat mass; QUICKI—quantitative insulin sensitivity check index.

FIGS. 3 and 4 demonstrate the relationship with measures of quality of cardiometabolic function as a function of the daily steps normalized to body fat mass. The two proxy variables for cardiometabolic function (TC:HDL ratio for FIG. 3 and QUICKI for FIG. 4) show significant correlation with daily steps taken per kilogram of fat mass indicated by R² values above 0.1.

A key finding of this research study was the strong relationship between STEPS expressed relative to FM (e.g., STEPS·FM⁻¹·day⁻¹) and markers of body fatness. While a number of cross-sectional studies have reported inverse relationships between daily step counts and body fatness when STEPS are reported in absolute terms (e.g., STEPS·day⁻¹), results of the current study yielded no such relationship (see Table 2 and FIG. 2). This may be due to differences in characteristics of samples (participants were men and women under the age of 40 years), or to differences in how step counts were derived (accelerometers vs pedometers). However, the power regression model depicted in FIG. 1 shows that daily step counts expressed relative to fat mass in samples explained 82% and 92% of the variability in BF in women and men, respectively, and that the slope of the relationship between STEPS·FM⁻¹·day⁻¹ and body composition was steepest among those with the greatest proportion of body fat.

The strength of the relationship between STEPS·FM⁻¹·day⁻¹ and body composition in adults eating ad libitum may be largely explained by recent studies reporting that, of a number of factors that have been shown to transiently influence energy intake, fat-free mass exerts a tonic (that is, constant, stable, in contrast to transience influences like incretins and adipokines) influence on appetite, and is strongly correlated with resting metabolic rate which accounts for over half of the variability in energy intake in adults. As an illustrative example, at any body weight, the dose of PA to achieve energy balance may account for the tonic influence of fat-free mass on appetite and energy intake. Furthermore, given that fat-free mass in an individual is quite stable with daily variations in PA, the tonic influence of fat-free mass on appetite and energy intake would be expected to make it difficult to maintain energy balance or a sustained energy deficit at very low levels of PA, as has been reported in several studies.

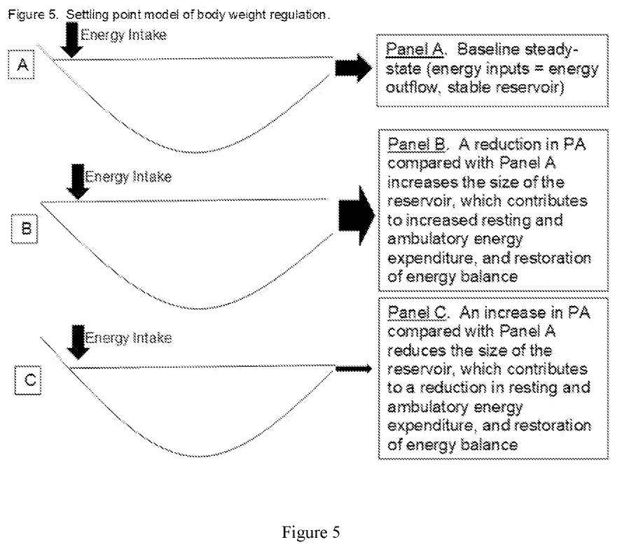

FIG. 5 shows a settling point model of body weight regulation.

This explanation aligns well with the “settling point” model of body weight (see FIG. 5). The model suggests that body weight “settles” at a level that results in an energy balance at any given energy intake and amount of PA. In application, in adults eating ad libitum, increases in PA that result in an energy deficit would be expected to result in a reduction in the “energy reservoir” represented by fat mass, which would lead to a reduction in resting and ambulatory energy expenditure and a restoration of energy balance. Likewise, a reduction in PA that leads to an energy surplus would be expected to induce an increase in body fatness, and consequently, an increase in resting and ambulatory energy expenditure resulting in restoration of energy balance.

Expressions of STEPS relative to fat mass appear to account for the influence of each element of the “settling point” model (i.e., energy intake, energy expenditure, and the energy reservoir represented by body fatness). For example (see Table 4), Subject A is a male weighing 80 kg with 25% body fat, and therefore has a lean mass of 60 kg, and a fat mass of 20 kg. Subject B is a male weighing 100 kg with 20% body fat, and has a lean mass of 80 kg and, like Subject A, also has a fat mass of 20 kg. The power regression model resulting from analysis of data from a pilot study (see FIG. 1) predicts an accumulation of 710 steps·kg of fat mass⁻¹·day⁻¹ for the heavier, leaner individual (14,199 steps. day⁻¹), but 526 steps·kg of fat mass⁻¹day⁻¹ in the lighter, less-lean individual (10,580 steps·day⁻¹). Therefore, while they possess the same amount of fat mass, the individual with a greater fat-free mass (Subject B) accumulates a greater number of average daily steps (3,619 steps·day' more according to a regression model) than the lighter person (Subject A) to achieve energy balance while eating ad libitum. The additional steps counter the influence of a greater amount of fat-free mass on tonic appetite and energy intake, partially compensated by the increased resting metabolic rate and energy cost of ambulation in the heavier individual.

TABLE 4
STEPS with Equal Fat Mass but Different
Body Composition (Males).

| | Subject A | Subject B |
|---|---|---|
| Weight (kg) | 80 | 100 |
| Body Fat Percentage | 25 | 20 |
| Fat Mass (kg) | 20 | 20 |
| Fat Free Mass (kg) | 60 | 80 |
| Steps · kg of fat mass⁻¹ · day⁻¹ | 526 | 710 |
| Total Steps · day⁻¹ | 10,580 | 14,199 |

In another example wherein two women weigh the same, but have different body compositions, (see Table 5), Subject C accumulates ˜1,332 steps/day more than Subject D, resulting in body fat mass “settling” at 24 vs 28 kg. The additional PA that Subject C accumulates may serve to compensate for the increased appetite and energy intake stimulated by the larger fat-free mass, while also increasing total daily energy expenditure through PA, thereby leading to a lower settling point for body fatness.

TABLE 5
STEPS with Equal Weight but Different Fat Mass (Females).

| | Subject C | Subject D |
|---|---|---|
| Weight (kg) | 80 | 80 |
| Body Fat Percentage | 30 | 35 |
| Fat Mass (kg) | 24 | 28 |
| Fat Free Mass (kg) | 56 | 52 |
| Steps · kg of fat mass⁻¹ · day⁻¹ | 437 | 327 |
| Total Steps · day⁻¹ | 10,488 | 9,156 |

While an interventional study was not conducted, the strength of this relationship suggests that it may be possible to prescribe PA specific to current and target body composition to produce predictable changes in body fatness in adults eating ad libitum. For example, in a weight management application, a man weighing 80 kg with 25% body fat possesses 20 kg of fat. Utilizing the results of power regression analysis, BF =2679.5·(STEPS·kg FM⁻¹·day⁻¹)^{-0.746} results in a predicted STEP count of approximately 526.4 STEPS·kg FM⁻¹·day⁻¹, or about 10,528 STEPS·day⁻¹. Assuming a target body composition of 20% fat, with the FM that he currently possesses, this individual may be prescribed a target of 709.91 STEPS·kg FM⁻¹·day⁻¹ (relative step counts associated with 20% BF), or about 14,199 STEPS·day^{31 1}.

A prescribed dose of PA, and particularly STEP counts, relative to an individual's current fat mass and targeted body composition, may be effective in identifying a PA target to result in a predictable energy deficit. Currently, the American College of Sports Medicine (ACSM) and other professional organizations express recommended exercise doses in absolute terms (e.g., kcals/week, steps/day). For example, ACSM recommends the accumulation of ≥2,000 kcal of exercise per week by those for whom weight loss/management is a priority. Similarly, the National Weight Control Registry has recently recommended the accumulation of 2,500-2,800 kcal of activity per week for weight control. However, as noted previously, a number of studies have reported that PA/exercise produces unpredictable and disappointing results for weight management relative to dietary restrictions, suggesting that the PA interventions were inadequate to induce an energy deficit. Findings suggest that expressions of PA as STEPS relative to FM are capable of predicting body composition, and may be efficacious for prescribing PA for achieving weight management objectives in adults eating ad libitum.

Physical Activity and Cardiometabolic Risk Factors

As reflected in the diagnostic criteria for metabolic syndrome, excess body fatness is linked with unfavorable CMR profiles (e.g., fasting blood glucose, dyslipidemia). However, at any given level of body fatness, CMR status may be improved via increases in PA. The findings of strong associations between expressions of PA and CMR factors are supportive of the capacity for PA to improve CMR status at any given level of body fatness. As shown in Table 6, and in FIGS. 3 and 4, expressions of PA were predictive of HDL, TC:HDL, fasting GLU, and QUICKI, particularly when PA was expressed as STEPS·kg FM⁻¹·day⁻¹. For example (See Table 6), the regression model shown in FIG. 3 shows that a man (Subject E) weighing 100 kg with 25% body fat and a TC:HDL ratio of 4.5 accumulates approximately 431 STEPS·kg FM⁻¹·day⁻¹, or a total of approximately 10,775 STEPS day⁻¹, while another man (Subject F) with the same weight and body fatness, but a healthier TC: HDL ratio of 4.0 accumulates approximately 633.3 STEPS·kg FM⁻¹·day⁻¹, or a total of approximately 15,833 STEPS·day⁻¹. Furthermore, although increasing daily step counts by such a margin may result in weight loss, the model suggests merely increasing physical activity may lead to improved CMR profiles without the requirement of decreasing FM.

TABLE 6
STEPS and Cardiometabolic Risk (Males).

| | Subject E | Subject F |
|---|---|---|
| Weight (kg) | 100 | 100 |
| Body Fat Percentage | 25 | 25 |
| Fat Mass (kg) | 25 | 25 |
| Fat Free Mass (kg) | 75 | 75 |
| TC:HDL Ratio | 4.5 | 4.0 |
| Steps · kg of fat mass⁻¹ · day⁻¹ | 431 | 633 |
| Total Steps · day⁻¹ | 10,775 | 15,833 |

TC = total cholesterol;
HDL = high-density lipoprotein cholesterol

In summary, step counts expressed relative to FM were strongly predictive of body composition in adults, a finding that suggests that physical activity prescribed based on current and target body composition may be efficacious in yielding predictable changes in body fatness in adults eating ad libitum. Similarly, physical activity expressed relative to FM was strongly predictive of several common markers of CMR, suggesting that levels of CMR factors may be predictably affected by changes in physical activity expressed relative to FM.

The systems and methods described herein utilized accelerometers worn on the non-dominant wrist to record physical activity and step counts. The wrist was chosen instead of the hip because participants were to be wearing the accelerometers for at least 21 days, and many had indicated a reluctance to wear the device on the hip due to appearance, a preference that others have previously reported as well. In addition, while estimates of physical activity energy expenditure were recorded using the Actigraph accelerometers, others have recently reported concerns with validity of energy expenditure estimates derived from these devices. The systems and methods described herein, therefore, limited analysis in this study to STEPS, MVPA, and SED. It is noteworthy, furthermore, that expressions of STEPS were more predictive of CMR and body composition outcomes than estimates of physical activity energy expenditure.

A further study was conducted for the purposes of determining the efficacy of the model reported previously developing daily step count targets that may be expected to produce predictable changes in body weight and body fatness in adults 19-40 years of age eating ad libitum. To date, we have had six participants (29.3±5.7 years old, 166.6±9.2 cm tall), all women, undergo two assessments of body weight and composition. Assessments were separated by three months, and activity trackers (e.g., pedometers were worn daily for those three months. After the first assessment, the model was used to develop a step count target predicted to yield a 7% weight loss, and participants were encouraged to strive to achieve those targets as regularly as possible. Daily step counts were submitted approximately weekly, and participants were provided feedback on how they were doing relative to their targets on two occasions over three months. After three months of pedometer wear, participants underwent assessments of body composition identical to those conducted at baseline, and changes in weight and body fatness were analyzed. In addition, actual step counts (average over previous 4 weeks) were compared with target step counts, and actual step counts were applied to the model to predict body composition.

TABLE 7
Measures of body weight and composition.

| | Baseline | 3-months | t | p |
|---|---|---|---|---|
| Weight (kg) | 92.0 ± 13.4 | 91.3 ± 14.4 | 0.56 | 0.602 |
| BMI (kg · m⁻²) | 33.2 ± 5.0 | 33.0 ± 5.4 | 0.57 | 0.594 |
| Body Fatness (%) | 40.4 ± 7.5 | 39.2 ± 7.7 | 1.71 | 0.147 |
| Fat Mass (kg) | 37.9 ± 11.7 | 36.6 ± 11.8 | 1.19 | 0.289 |
| Fat-Free Mass (kg) | 54.1 ± 4.7 | 55.4 ± 5.3 | 2.02 | 0.100 |
| Visceral Fatness (cc) | 586.3 ± 302.9 | 558.5 ± 297.4 | 2.10 | 0.090 |

t = correlated t-test statistic;
p = probability

As shown in Table 7, none of the changes in weight or body fatness were significant at 3-months, though all of the means improved.

Although participants were walking more at 3-months than they were at the onset of the study, they were coming up short of their daily target by an average of ˜1,850 steps per day (see Table 8).

TABLE 8
Actual and target daily step counts.

| | Target | Actual | t | p |
|---|---|---|---|---|
| Steps/day | 11525 ± 1638 | 9672 ± 1486 | 2.02 | 0.10 |

t = correlated t-test statistic;
p = probability

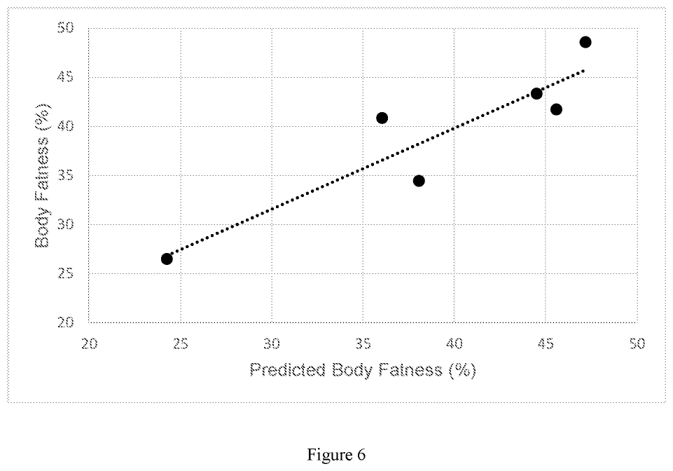

Application of the model to actual step counts resulted in predictions of body fatness that were strongly correlated with actual body composition (see Table 9 and FIG. 6).

TABLE 9
Actual vs predicted body composition.

| | Predicted | Actual | t | p | r | p |
|---|---|---|---|---|---|---|
| Body Fatness (%) | 39.3 ± 8.6 | 39.2 ± 7.7 | 0.05 | 0.965 | 0.92 | 0.01 |

t = correlated t-test statistic;
r = Pearson produce moment correlation;
p = probability

These results support the efficacy of the model for developing step count targets that, if adhered to, may be expected to result in predictable changes in body weight and composition.

Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. Thus, it is impossible for any program to damage the execution of any of the other programs running on the system. However, the programs have no way to exchange any information except through the operating system (or by reading files stored on the file system).

Multi-process computing is similar to multi-tasking computing, as the terms task and process are often used interchangeably, although some operating systems make a distinction between the two. The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing.

A computer readable storage medium, as used herein, is not to be construed as being transitory signals per se, such as radio waves or other freely propagating electromagnetic waves, electromagnetic waves propagating through a waveguide or other transmission media (for example, light pulses passing through a fiber-optic cable), or electrical signals transmitted through a wire. Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s).

In some alternative implementations, the functions noted in the blocks may occur out of the order noted in the Figures. For example, two blocks shown in succession may, in fact, be executed substantially concurrently, or in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or that carry out combinations of special purpose hardware and computer instructions. Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

From the above description, it can be seen that the present invention provides a system, computer program product, and method for the efficient execution of the described techniques. References in the claims to an element in the singular is not intended to mean “one and only” unless explicitly so stated, but rather “one or more.” All structural and functional equivalents to the elements of the above-described exemplary embodiment that are currently known or later come to be known to those of ordinary skill in the art are intended to be encompassed by the present claims. No claim element herein is to be construed under the provisions of 35 U.S.C. section 112, sixth paragraph, unless the element is expressly recited using the phrase “means for” or “step for.”

While the foregoing written description of the invention enables one of ordinary skill to make and use what is considered presently to be the best mode thereof, those of ordinary skill will understand and appreciate the existence of alternatives, adaptations, variations, combinations, and equivalents of the specific embodiment, method, and examples herein. Those skilled in the art will appreciate that the within disclosures are exemplary only and that various modifications may be made within the scope of the present invention. In addition, while a particular feature of the teachings may have been disclosed with respect to only one of several implementations, such feature may be combined with one or more other features of the other implementations as may be desired and advantageous for any given or particular function. Furthermore, to the extent that the terms “including”, “includes”, “having”, “has”, “with”, or variants thereof are used in either the detailed description and the claims, such terms are intended to be inclusive in a manner similar to the term “comprising”.

Other embodiments of the teachings will be apparent to those skilled in the art from consideration of the specification and practice of the teachings disclosed herein. The invention should therefore not be limited by the described embodiment, method, and examples, but by all embodiments and methods within the scope and spirit of the invention. Accordingly, the present invention is not limited to the specific embodiments as illustrated herein, but is only limited by the following claims.

What is claimed is:

1. A method for customized activity level recommendations, the method comprising:
receiving via an interface, at least one body health indicator associated with a user;
determining, based at least on the body health indicator, a fat mass metric of the user and a weight/body fatness target of the user;
determining, based on at least the fat mass metric and using a recommendation algorithm comprising a set of rules, a custom physical activity threshold for the user; and
outputting an indication of the custom physical activity threshold.

2. The method of claim 1 wherein the at least one body health indicator comprises user sex, body mass, body fat mass, fat-free mass, body fatness percentage, visceral fat mass, total cholesterol level, high-density lipoprotein cholesterol level, low-density lipoprotein cholesterol level, insulin level, glucose level, triglyceride level, glycosylated hemoglobin level, a cardiometabolic risk factor, an energy intake, an energy expenditure or combinations thereof.

3. The method of claim 1, wherein the at least one body health indicator comprises a user's target body mass, target body fat mass, target fat-free mass, target body fatness percentage, target visceral fat mass, target total cholesterol level, target high-density lipoprotein cholesterol level, target low-density lipoprotein cholesterol level, target insulin level, target glucose level, target triglyceride level, target glycosylated hemoglobin level, target cardiometabolic risk factor, target energy intake, or target energy expenditure or some combination thereof.

4. The method of claim 1, wherein the custom physical activity threshold comprises a number of steps taken per day.

5. The method of claim 1, wherein the custom physical activity threshold comprises a number of steps taken per day per unit of body fat mass, and based thereon, a total daily step count.

6. The method of claim 1, wherein the interface comprises an activity tracking device.

7. A non-transitory computer readable storage medium having instructions stored thereon that, in response to execution by a computing device, causes the computing device to execute a method for customized activity level recommendations, the method comprising:
receiving via an interface, at least one body health indicator associated with a user;
determining, based at least on the body health indicator, a fat mass metric of the user and a weight/body fatness target of the user;
determining, based on at least the fat mass metric and using a recommendation algorithm comprising a set of rules, a custom physical activity threshold for the user; and
outputting an indication of the custom physical activity threshold.

8. The non-transitory computer readable storage medium of claim 7, wherein the at least one body health indicator comprises user sex, body mass, body fat mass, fat-free mass, body fatness percentage, visceral fat mass, total cholesterol level, high-density lipoprotein cholesterol level, low-density lipoprotein cholesterol level, insulin level, glucose level, triglyceride level, glycosylated hemoglobin level, a cardiometabolic risk factor, an energy intake, an energy expenditure or combinations thereof.

9. The non-transitory computer readable storage medium of claim 7, wherein the at least one body health indicator comprises a user's target body mass, target body fat mass, target fat-free mass, target body fatness percentage, target visceral fat mass, target total cholesterol level, target high-density lipoprotein cholesterol level, target low-density lipoprotein cholesterol level, target insulin level, target glucose level, target triglyceride level, target glycosylated hemoglobin level, target cardiometabolic risk factor, target energy intake, or target energy expenditure or some combination thereof.

10. The non-transitory computer readable storage medium of claim 7, wherein the custom physical activity threshold comprises a number of steps taken per day.

11. The non-transitory computer readable storage medium of claim 7, wherein the custom physical activity threshold comprises a number of steps taken per day per unit of body fat mass, and based thereon, a total daily step count.

12. The non-transitory computer readable storage medium of claim 7, further comprising, causing the computing device to execute a recommendation algorithm using at least one body health indicator and providing a custom physical activity threshold.

13. The non-transitory computer readable storage medium of claim 7, wherein the interface comprises an activity tracking device.

14. A device for executing a method for customized activity level recommendations, the device comprising:
an interface configured to:
receive at least one body health indicator associated with a user;
determine, based at least on the body health indicator, a fat mass metric of the user and a weight/body fatness target of the user;
determine, based on at least the fat mass metric and using a recommendation algorithm comprising a set of rules, a custom physical activity threshold for the user; and
output an indication of the custom physical activity threshold.

15. The device of claim 14, wherein the interface comprises an activity tracking device.

16. The device of claim 14, wherein the at least one body health indicator comprises user sex, body mass, body fat mass, fat-free mass, body fatness percentage, visceral fat mass, total cholesterol level, high-density lipoprotein cholesterol level, low-density lipoprotein cholesterol level, insulin level, glucose level, triglyceride level, glycosylated hemoglobin level, a cardiometabolic risk factor, an energy intake, an energy expenditure or combinations thereof.

17. The device of claim 14, wherein the at least one body health indicator comprises a user's target body mass, target body fat mass, target fat-free mass, target body fatness percentage, target visceral fat mass, target total cholesterol level, target high-density lipoprotein cholesterol level, target low-density lipoprotein cholesterol level, target insulin level, target glucose level, target triglyceride level, target glycosylated hemoglobin level, target cardiometabolic risk factor, target energy intake, or target energy expenditure or some combination thereof.

18. The device of claim 14, wherein the interface wherein the custom physical activity threshold comprises a number of steps taken per day.

19. The device of claim 14, wherein the custom physical activity threshold comprises a number of steps taken per day per unit of body fat mass, and based thereon, a total daily step count.

20. The device of claim 14, wherein the interface is further configured to execute a recommendation algorithm using at least one body health indicator and providing a custom physical activity threshold.
